(19) United States
(12) Patent Application Publication
(10) Pub. No.: US 2026/0363693 A1
(43) Pub. Date: Nov. 19, 2026

(54) ANATOMICAL STRUCTURE APPROXIMATING DEVICES, SYSTEMS, AND METHODS

(71) Applicant: Boston Scientific Scimed, Inc., Maple Grove, MN (US)

(72) Inventor: Timothy Noah Foster-Frau, Austin, TX (US)

(73) Assignee: Boston Scientific Scimed, Inc., Maple Grove, MN (US)

(21) Appl. No.: 19/680,025

(22) Filed: May 18, 2026

Related U.S. Application Data
(60) Provisional application No. 63/808,228, filed on May 19, 2025.

(51) Int. Cl. A61B 17/04; A61B 1/018; A61B 17/06; A61B 17/00

(52) U.S. Cl. CPC A61B 17/0401; A61B 1/018; A61B 17/0469; A61B 17/06166; A61B 2017/00477; A61B 2017/00862; A61B 2017/0409; A61B 2017/0414; A61B 2017/0416; A61B 2017/0441; A61B 2017/0462; A61B 2017/0479; A61B 2017/0618

(57) ABSTRACT

A system for approximating, such as drawing together, anatomical structure(s) such as tissue. The system includes two or more tissue engagement members which are operably coupled with a flexible elongate member, such as a suture. The flexible elongate member is shiftable between a configuration allowing relative movement between the tissue engagement members and the flexible elongate member, and a configuration in which the tissue engagement members are held in place with respect to the flexible elongate member. With the tissue engagement members held in place with respect to the flexible elongate member, the tissue engagement members are held together with respect to one another and/or the anatomical structure(s) engaged by the tissue engagement members is approximated or cinched or otherwise drawn together.

### CROSS-REFERENCE TO RELATED APPLICATIONS

This application claims the benefit of priority under 35 U.S.C. § 119 to U.S. Provisional Application No. 63/808,228, filed on May 19, 2025, the entire disclosure of which is hereby incorporated by reference herein for all purposes.

### FIELD

The present disclosure relates generally to the field of medical devices, systems, and method for closing or approximating or otherwise drawing together anatomical structures such as tissue. In particular, the present disclosure relates to medical devices, systems, and methods for improving closure or approximation achieved with an elongate flexible element such as a suture.

### BACKGROUND

Various medical procedures involve closing, approximating, gathering, stitching, suturing, stapling, or otherwise drawing together anatomical structures. For instance, various procedures involve closing open wounds, such as after tissue resection or dissection. Typically, a flexible elongate member, such as a suture, is used to draw together the tissue to close the undesired separation or opening between tissue edges. Additionally or alternatively, hemostasis clips may be used to provide a compressive force to hold together anatomical structures such as tissue. Hemostasis clips may be used at the site of a bleed to cause hemostasis in a quick and efficient manner. However, some clips may be difficult to position with precision with respect to a lesion, and/or once “fired”, may lack the ability to be removed and repositioned. Since most clips for internal use (e.g., transluminal, such as endoscopic, delivery and deployment) are relatively small and present a limited surface area which may act on only on a localized area of tissue, multiple clips may be used to achieve the desired treatment effect. In some instances, tissue anchors may be deployed into tissue and pulled together with a flexible elongate member, such as a suture, to draw together the tissue. In any solution involving use of a suture, a cinch may be used to pull the suture tight, and a plug may be deposited to prevent loosening of the suture. In the case of a suture used in conjunction with tissue anchors, a plug may be used to prevent the suture from being pulled out of the anchors. As may be appreciated, tensioning of the suture is accompanied by the risk of inadvertently breaking the suture. Also, when properly used, a few millimeters of suture remains in the patient along with the cinch plug. It is with respect to these and other considerations and challenges that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, in accordance with various principles of the present disclosure, a system for approximating anatomical structures includes two or more tissue engagement members each having a distal end configured to be secured to an anatomical structure, and a proximal end; and a flexible elongate member operably coupled with said two or more tissue engagement members. In some aspects, the two or more tissue engagement members include a distalmost tissue engagement member and one or more tissue engagement members proximal to the distalmost tissue engagement member along the flexible elongate member; and at least a portion of said flexible elongate member is shiftable between a first configuration in which said one or more tissue engagement members proximal to said distalmost tissue engagement member are operably coupled yet inhibited from moving longitudinally with respect to said flexible elongate member, and a second configuration in which said one or more tissue engagement members proximal to said distalmost tissue engagement member are operably coupled and longitudinally movable with respect to said flexible elongate member.

In some aspects, the shiftable portion of the flexible elongate member is a distal portion of the flexible elongate member.

In some aspects, the shiftable portion of the flexible elongate member extends through a passage defined through a portion of one or more of the tissue engagement members. In some aspects, in the first configuration, the shiftable portion of the flexible elongate member has a larger-diameter relaxed configuration extending through the passage to inhibit relative movement between the one or more of the tissue engagement members and the flexible elongate member; and in the second configuration, the shiftable portion of the flexible elongate member has a smaller-diameter extended configuration smaller than the passage defined through the one or more of the tissue engagement members to allow longitudinal movement of the one or more tissue engagement members with respect to the flexible elongate member in the second configuration. In some aspects, one or more of the tissue engagement members includes a coupler, the passage being defined through the coupler. In some aspects, the coupler of at least one of the one or more of the tissue engagement members is rotatably coupled with respect the at least one of the one or more of the tissue engagement members.

In some aspects, the shiftable portion is elastomeric.

In some aspects, the distalmost tissue engagement member is fixedly coupled to a distalmost end of the flexible elongate member so as not to move longitudinally with respect to the flexible elongate member.

In some aspects, the two or more tissue engagement members are rotatable about the flexible elongate member.

In some aspects, at least one of the tissue engagement members is a helical tissue anchor. In some aspects, the helical tissue anchor includes a coupler rotatably coupled thereto and extending radially from the helical tissue anchor to be operably coupled with the flexible elongate member.

In some aspects, in accordance with various principles of the present disclosure, a system for approximating anatomical structures includes two or more tissue engagement members each having a distal end configured to be secured to an anatomical structure, and a proximal end; a flexible elongate member operably coupled with the two or more tissue engagement members; and a delivery device configured to deliver the two or more tissue engagement members and the flexible elongate member to a treatment site within a patient. In some aspects, the two or more tissue engagement members include a distalmost tissue engagement member and one or more tissue engagement members proximal to the distalmost tissue engagement member; and at least a portion of the flexible elongate member is shiftable between a first configuration in which the one or more tissue engagement members proximal to the distalmost tissue engagement member are operably coupled yet inhibited from moving longitudinally with respect to the flexible elongate member, and a second configuration in which the one or more tissue engagement members proximal to the distalmost tissue engagement member are operably coupled and longitudinally movable with respect to the flexible elongate member.

In some aspects, the system further includes a medical scope, the delivery device having a flexible tubular elongate member deliverable with a flexible distal end of the medical scope into a patient and advanceable within the patient to a treatment site.

In some aspects, the one or more tissue engagement members are helical anchors with a sharp distalmost end configured to penetrate tissue, and the delivery device is configured to engage each of the tissue engagement member to rotate each of the tissue engagement members into engagement with tissue and to distally push each of the tissue engagement members out of engagement with the delivery device to deploy the tissue engagement member. In some aspects, in the first configuration, the portion of the flexible elongate member is in a relaxed configuration, and after deployment of all of the two or more tissue engagement members by the delivery device, the portion of the flexible elongate member may be cut and left in the first configuration with all of the tissue engagement members inhibited from moving longitudinally with respect to the flexible elongate member.

In some aspects, a method of drawing one or more anatomical structures together in accordance with various principles of the present disclosure includes engaging a first tissue engagement member with the anatomical structure at a first deployment site and deploying the first tissue engagement member; shifting a flexible elongate member, operably coupled with the first tissue engagement member, from a first configuration to a second configuration; advancing a second tissue engagement member distally along the flexible elongate member and towards the first tissue engagement member; engaging the second tissue engagement member with the anatomical structure, or another anatomical structure, at a second deployment site spaced apart from the first deployment site and deploying the second tissue engagement member; cinching the flexible elongate member to draw together the first and second tissue engagement members as well as the deployment sites; and shifting at least a portion of the flexible elongate member to the first configuration to retain positions of the deployed tissue engagement members with respect to the flexible elongate member.

In some aspects, the first configuration of the flexible elongate member is a relaxed configuration, the method further comprising releasing force on the flexible elongate member to allow the flexible elongate member to shift to the first configuration to hold the deployed anchors in place with respect to the flexible elongate member.

In some aspects, the method further includes holding the deployed anchors in place with respect to the anatomical structure and to one another by allowing the flexible elongate member to shift to its first configuration, thereby drawing together the one or more anatomical structures.

In some aspects, the method further includes severing an extent of the flexible elongate member proximal to the deployed tissue engagement members. In some aspects, a portion of the flexible elongate member operably coupled with the deployed tissue engagement members is left in the first configuration maintaining drawn together positions of the deployed tissue engagement members with respect to the flexible elongate member.

In some aspects, the method further includes advancing one or more additional tissue engagement members distally along the flexible elongate member with the flexible elongate member in the second configuration; engaging the one or more additional tissue engagement members with respect to the anatomical structure, or another anatomical structure, at one or more further deployment sites spaced apart from the first deployment site and the second deployment site; deploying the one or more additional tissue engagement members; cinching the flexible elongate member to draw together the one or more additional tissue engagement members and the first and second tissue engagement members as well as the respective deployment sites; and shifting at least a portion of the flexible elongate member to the first configuration to retain positions of the deployed tissue engagement members with respect to the flexible elongate member.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope for transluminal delivery within a patient’s body. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an example of an embodiment of a medical system formed in accordance with various principles of the present disclosure for closing or approximating anatomical structures.

FIG. 2 illustrates an example of an embodiment of an instrument of a system such as illustrated in FIG. 1.

FIG. 3 illustrates an example of an embodiment of a system for engaging and drawing together the engaged anatomical structure.

FIGS. 4A and 4B illustrate, schematically, advancement of anchors along a flexible elongate member in accordance with various principles of the present disclosure.

FIGS. 5A-5H illustrate, schematically, deployment and cinching of a system of anchors and a flexible elongate member such as illustrated in FIGS. 4A and 4B.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

In accordance with various principles of the present disclosure, two or more anatomical engagement elements are engaged with an anatomical structure at a treatment site, and operably coupled together with a flexible elongate member to draw together portions of the anatomical structure at a treatment site. It will be appreciated that reference to an anatomical structure is intended to include reference to different portions of an anatomical structure and/or reference to more than one anatomical structure. Additionally, it will be appreciated that reference may be made to a treatment site, anatomical site, delivery site, deployment site, implant / implantation site, site of implantation, target site, etc., interchangeably herein or with other variations which may be appreciated by those of ordinary skill in the art and without intent to limit. For the sake of simplicity, and without intent to limit, reference is made to engagement of a tissue engagement member with respect to tissue, it being appreciated that principles of the present disclosure have broader applicability. It will be appreciated that terms such as engage, secure, anchor, couple, grasp, hold, join, clamp, clasp, clip, attach, affix, associate, deploy on, etc. (and other grammatical forms thereof) may be usable interchangeably herein without intent to limit. Reference is made to a tissue engagement member for the sake of convenience, and without intent to limit, and reference may be made even more simply to a tissue anchor without intent to limit, it being appreciated that principles of the present disclosure have broader applicability. Also, for the sake of simplicity, and without intent to limit, reference is made to a flexible elongate member or suture, it being appreciated that principles of the present disclosure have broader applicability. The flexible elongate member is operably coupled with respect to the tissue engagement members so that pulling or cinching of the flexible elongate member draws together the tissue engagement members to draw together the deployment sites at which the anchors have been deployed (e.g., anatomical structure(s) with respect to which the tissue engagement members are engaged).

In accordance with various principles of the present disclosure, at least a portion (e.g., a distal portion) of the flexible elongate member is shiftable between a first configuration and a second configuration. In the first configuration of flexible elongate member, the flexible elongate member may be considered to be relaxed, without external forces applied thereto, and the tissue engagement members are operably coupled with respect to the flexible elongate member in a substantially fixed longitudinal position along the flexible elongate member. More particularly, when the flexible elongate member is in the first configuration, the tissue engagement members are held in place longitudinally so that the tissue engagement members do not slide along the flexible elongate member. It will be appreciated that in some instances a sufficient force may be applied to cause sliding of the tissue engagement members with respect to the flexible elongate member, but, during use, relative movement between the tissue engagement members and the flexible elongate member is inhibited and substantially prevented. In some aspects, only longitudinal movement of the tissue engagement members is inhibited, and at least some degree of rotational movement is possible. The anatomical structure being approximated with the use of the tissue engagement members and flexible elongate member is thus held in approximation by the flexible elongate member (in its first configuration) and the tissue engagement members operably coupled to the flexible elongate member and engaged with the anatomical structure. In the second configuration of the flexible elongate member, the tissue engagement members (at least the tissue engagement members proximal to the distalmost, first deployed tissue engagement members) are movable (e.g., slidably, longitudinally translatable, etc.) along the flexible elongate member (e.g., along the longitudinal axis of the flexible elongate member). Such movability of the tissue engagement members along the flexible elongate member may allow for adjustment of the relative positions of the tissue engagement members with respect to one another as well as with respect to the flexible elongate member and optionally also with respect to the anatomical structure with respect to which the tissue engagement members are engaged or are to be engaged. In some aspects, the distalmost, first deployed tissue engagement member is substantially fixed to the flexible elongate member, and generally does not move along the length of the flexible elongate member. It will be appreciated that some or all of the tissue engagement members may be rotatable with respect to the flexible elongate member (e.g., about the longitudinal axis of the flexible elongate member) and with respect to one another. Additionally or alternatively, some or all of the tissue engagement members may not be linked rotationally.

In some aspects, one or more of the tissue engagement members include a coupler with respect to which the flexible elongate member may be operably coupled with one or more of the tissue engagement members. In some aspects, one or more of at least the tissue engagement members proximal to the leading, distalmost anchor (operably coupled with the distalmost end of the flexible elongate member) includes a passage through which the flexible elongate member may movably (e.g., slidably) extend. In some aspects, the passage is defined through a coupler of the tissue engagement member. The passage allows for the tissue engagement members to be advanced with respect to the flexible elongate member for placement with respect to anatomical structure. For instance, after deployment of the leading, distalmost tissue engagement member with respect to anatomical structure, additional tissue engagement members may be advanced distally over the flexible elongate member to an already-deployed tissue engagement member (which has been secured with respect to the anatomical structure at a selected deployment site). Each additional tissue engagement member which is advanced over the flexible elongate member may be deployed with respect to the anatomical structure to be secured with respect to the anatomical structure at such deployment site. The shiftable portion of the flexible elongate member shifts into a configuration which may interact with the coupler and/or passage of the anchor to maintain / retain a substantially fixed relative position of the flexible elongate member and anchor. Such interaction may be an interference fit, a friction fit, etc.

In some aspects, the portion of the flexible elongate member which shifts between configurations is a distal portion of the flexible elongate member. In some aspects, the portion of the flexible elongate member which is shiftable is configured to shift between an increased diameter configuration to hold the tissue engagement members in place with respect to the flexible elongate member, and a smaller diameter configuration allowing movement (e.g., sliding) of the tissue engagement members with respect to the flexible elongate member. In some aspects, the shiftable portion is formed of a flexible, stretchable, expandable material. For instance, the distal portion may be formed of an elastomeric material such as polypropylene or silicone, or any other suitable preferably biocompatible material. Additionally or alternatively, the distal portion may be formed of a spring element, such as a metal coil spring. In the smaller diameter configuration, the distal portion has a diameter smaller than the inner diameter of the passage through which the flexible elongate member extends so that the tissue engagement members may slide or longitudinally advance along the flexible elongate member. In the larger diameter configuration, the distal portion has a diameter larger than the inner diameter of the passage through which the flexible elongate member extends so that the tissue engagement members cannot readily move or slide with respect to the flexible elongate member. In some aspects, the distal portion of the flexible elongate member is shifted from the larger diameter configuration to the smaller diameter configuration by application of tension to (e.g., by pulling on) the flexible elongate member, such as to stretch the distal section thereof. When tension is relaxed or released, the distal portion returns to the larger diameter configuration, substantially trapping the tissue engagement members in place. In the case of a spring-type distal portion, the coils of the spring would trap the tissue engagement members in place when not expanded into a more elongated (and less coiled) configuration. In some aspects, the distal portion, when in the larger diameter configuration, maintains an interference or friction fit with respect to the tissue engagement members so that the tissue engagement members are secured in place with respect to the flexible elongate member and thus with respect to the anatomical structure with respect to which the tissue engagement members have each been deployed. The fixing of the tissue engagement members and flexible elongate member may render the use of a cinch fixing element such as cinch plug unnecessary.

Various embodiments of devices, systems, and methods for closing or approximating anatomical structures such as tissue will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element).

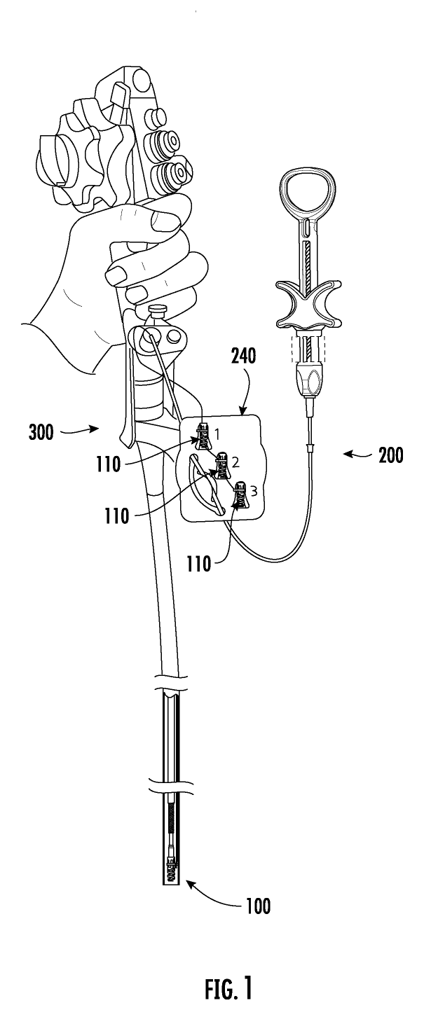

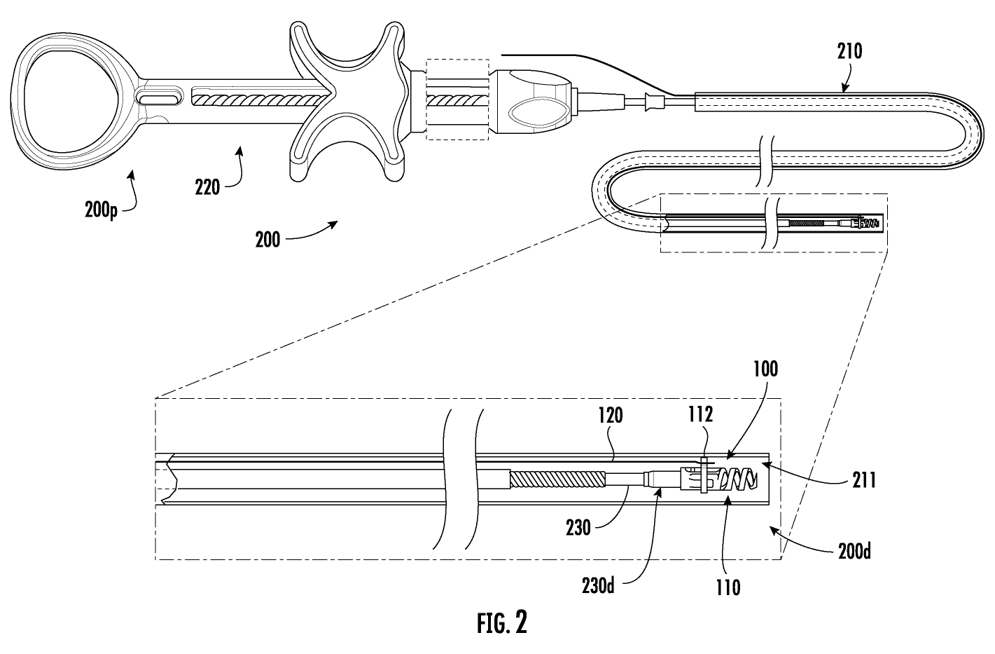

Turning now to the drawings, an example of an embodiment of a system 100 formed in accordance with various principles of the present disclosure to close or approximate or otherwise draw together anatomical structures is illustrated in FIGS. 1-3, 4A, 4B, and 5A-5H,. For the sake of convenience, and without intent to limit, the system 100 is referenced herein as a cinching system 100. The cinching system 100 may be delivered with or by a delivery device 200, as illustrated in FIGS. 1 and 2. For instance, the delivery device 200 may include a flexible tubular elongate member 210 with a lumen 211 defined therein configured to deliver the cinching system 100 therein, as may be appreciated with reference to FIG. 2. In some aspects, the flexible tubular elongate member 210 is sized for insertion into a patient through a natural orifice or small incision and is sufficiently flexible to be navigated within curved or tortuous pathways within a patient’s body to deliver the cinching system 100 within a patient’s body without the need for open surgery. In some aspects, the delivery device 200 includes a control handle 220, at a proximal end 200p of the delivery device 200, which may be used to control navigation of the flexible tubular elongate member 210 and/or deployment of the cinching system 100 out of the lumen 211 of the flexible tubular elongate member 210. Optionally, the cinching system 100 and the delivery device 200 are delivered with another medical device 300 configured to deliver one or more other medical devices (e.g., through one or more working channels defined through a flexible distal element of the medical device 300 which is insertable into the patient), irrigation fluid, vacuum pressure (suction), a visualization device (e.g., a camera, optic fiber, etc.), a light, etc. In some aspects, the medical device 300 is a medical scope, such as an endoscope, gastroscope, colonoscope, duodenoscope, laparoscope, ureteroscope, arthroscope, cystoscope, bronchoscope, hysteroscope, etc. In some aspects, the cinching system 100 along with the delivery device 200 and/or the medical device 300 may be considered to together form a medical closure or approximation system.

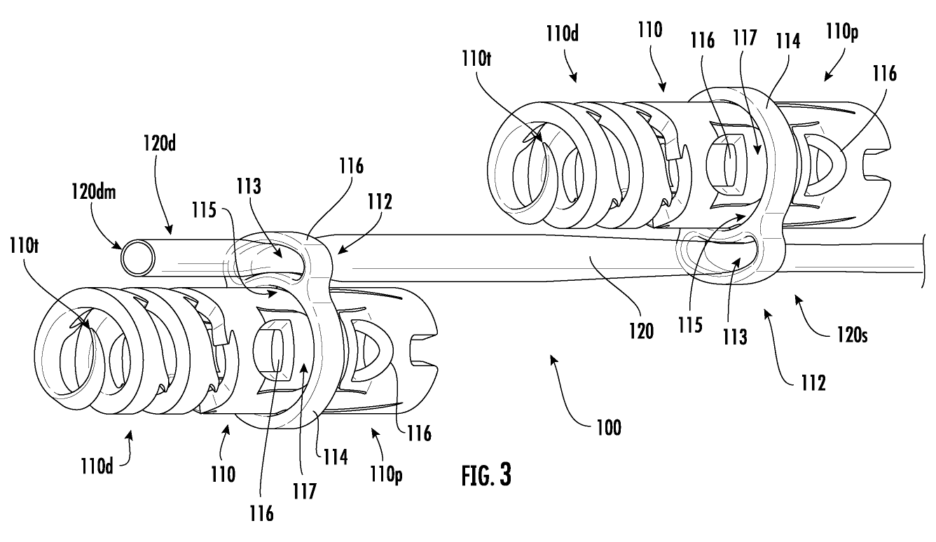

In accordance with various principles of the present disclosure, the cinching system 100 includes two or more anchors 110 and a flexible elongate member 120, as may be appreciated with reference to FIGS. 3, 4A, 4B and 5A-5H. The illustrated example of an embodiment of an anchor 110 is a helical anchor with a helically-formed distal end 110d ending in a sharp distal tip 110t (such as illustrated in FIG. 3) capable of penetrating tissue. However, it will be appreciated that the illustrated example of an embodiment is only one example of a tissue engagement member which may be used in accordance with various principles of the present disclosure, the present disclosure not necessarily limited to features of the anchor 110 described herein and illustrated in the accompanying figures. For instance, principles of the present disclosure are applicable to other configurations of tissue engagement members, such as tissue clips with a pair of jaws movable away from each other to receive tissue therebetween and towards each other to grasp tissue therebetween. Moreover, it will be appreciated that although reference is made to tissue, principles of the present disclosure apply to other anatomical structures as well.

In accordance with various principles of the present disclosure, the anchors 110 and flexible elongate member 120 of the cinching system 100 of the present disclosure are configured to be operably coupled together. In some aspects, such operably coupling allows selective relative movement between the anchors 110 and flexible elongate member 120, as well as fixing or retaining of relative positions of the anchors 110 with respect to the flexible elongate member 120 (e.g., maintaining a selected position of an anchor 110 along the longitudinal extent of the flexible elongate member 120). In some aspects, at least a portion or structure of one or more of the anchors 110 defines a passage 113 through which the flexible elongate member 120 may be extended to operably couple the flexible elongate member 120 with the anchor 110. The passage 113 may be an aperture, opening, hole, bore, channel, etc., sized, shaped, configured, and/or dimensioned for a portion of the flexible elongate member 120 to extend therethrough (either movably or substantially fixedly, as discussed in further detail below). In some aspects, one or more of the anchors 110 include a coupler 112 configured for coupling the anchor 110 with the flexible elongate member 120. In some aspects, such as in the example of an embodiment illustrated in FIG. 3, the coupler 112 defines the above-described passage 113 through which the flexible elongate member 120 may extend. It will be appreciated that the coupler 112 of the first of the anchors 110 to be deployed and engaged with an anatomical site (which may alternately be referenced as a leading or distalmost or first-deployed anchor 110) may simply be configured for the flexible elongate member 120 to be coupled thereto without necessarily passing therethrough. The flexible elongate member 120 may be coupled to the coupler 112 of the first of the anchors 110 to be deployed and engaged with an anatomical site in any of a variety of manners, such as may be selected based on the various properties of the anchor 110 and/or flexible elongate member 120. For instance, a portion (e.g., a distal end 120d, which may be a distalmost end 120dm) of the flexible elongate member 120 may tied and knotted, adhered (e.g., with a suitable adhesive), welded, overmolded, or otherwise secured with respect to the coupler 112 of the anchor 110. Optionally, an additional retainer, such as a band, a stopper lock (e.g., bead, bar, or other widened element which would prevent the distalmost end 110dm of the flexible elongate member 120 from being pulled out of a passage 113), a shrink wrap, or other separate structure, may be operably associated with flexible elongate member 120 and/or the coupler 112 (e.g., placed over the flexible elongate member 120 and/or the coupler 112), such as to secure the flexible elongate member 120 and coupler 112 of the distalmost anchor 110 together. In some aspects, the coupler 112 may be deformed (e.g., crimped) to secure the flexible elongate member 120 with respect thereto. It will be appreciated that other manners of fixedly securing (e.g., to prevent relative movement) at least a portion of the flexible elongate member 120 with at least the first of the anchors 110 to be deployed may be used without detracting from the principles of the present disclosure. It will further be appreciated that the flexible elongate member may be secured with respect to the distalmost anchor 110 via a structure other than a coupler and/or directly to the distalmost anchor 110. It will further be appreciated that a passage 113 may be defined in a structure of any of the anchors 110 other than a coupler 112.

In accordance with various principles of the present disclosure, at least a portion of the flexible elongate member 120 is shiftable between a first configuration, and a second configuration. In some aspects, in the first configuration, which may be considered a relaxed configuration, the flexible elongate member 120 and one or more of the anchors 110 are inhibited from moving with respect to each other, such as to maintain / retain the relative positions (e.g., longitudinal positions) of anchors 110 along the flexible elongate member 120. In the second configuration, the flexible elongate member 120 and the anchor 110 are movable with respect to each other, such as to allow the anchor 110 to advance distally along the flexible elongate member 120. In some aspects, the anchors 110 may rotate with respect to the flexible elongate member 120 when the flexible elongate member 120 is in the first configuration, but do not otherwise move longitudinally with respect to the flexible elongate member 120, such as to maintain a set position of the anchors 110 along the length of the flexible elongate member 120 and/or with respect to one another.

In some aspects, at least a portion of the flexible elongate member 120 is configured to shift between a larger-diameter relaxed configuration and a smaller-diameter extended configuration, such as illustrated in FIGS. 3, 4A, 4B, and 5A-5H. Optionally, the portion of the flexible elongate member 120 which shifts configurations is a distal portion 120d. As may be appreciated with reference to FIGS. 3, 4A, 4B and 5A-5H, in the first configuration, the larger-diameter relaxed configuration of the flexible elongate member 120 inhibits movement of the anchors 110 therealong (at least longitudinally). For instance, the larger-diameter relaxed configuration of the flexible elongate member 120 may be sufficiently large (e.g., larger than the inner dimensions of the passage 113 define through a portion of the anchor 110) to create a friction or interference fit with the passage 113 defined through a portion (e.g., a coupler 112) of the anchor 110 through which the flexible elongate member 120 extends. At least sliding movement of the anchor 110 along the length of the shiftable portion 120s of the flexible elongate member 120 may thereby be inhibited or prevented. In the second configuration, the flexible elongate member 120 is shifted to allow relative movement of the flexible elongate member 120 and the anchors 110. For instance, in the second configuration of the example of an embodiment of a flexible elongate member 120 illustrated in FIGS. 3, 4A, 4B, and 5A-5H, the shiftable portion 120s of the flexible elongate member 120 has a smaller-diameter extended configuration thin enough to slide or translate or otherwise move through the passage 113 defined through a portion (e.g., a coupler 112) of the anchor 110.

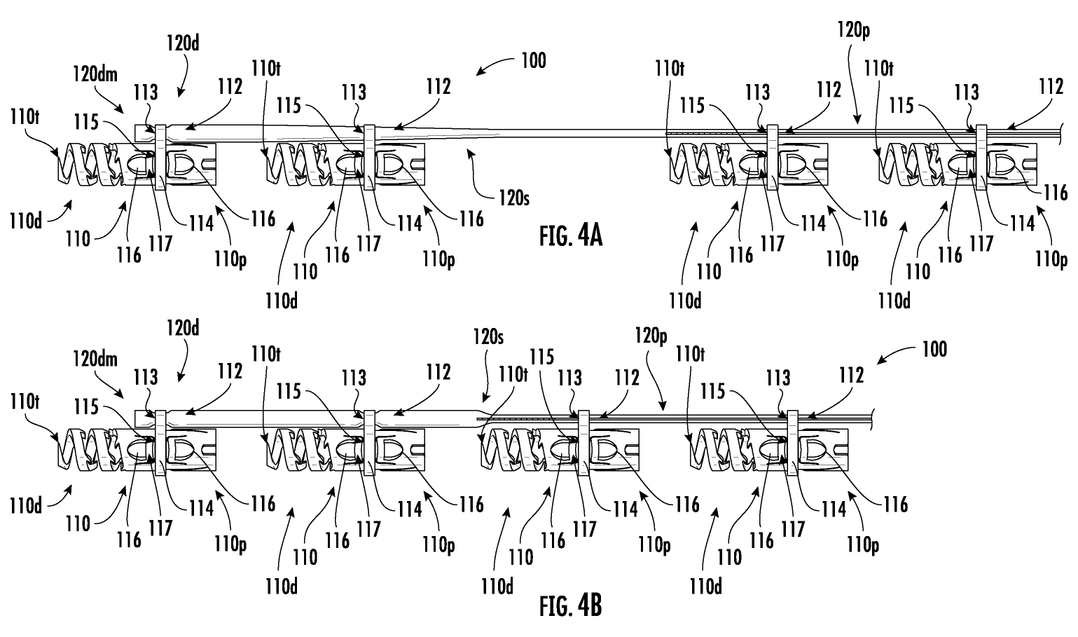

As may be appreciated upon comparison of the example of an embodiment illustrated in FIGS. 4A and 4B, the shiftable portion 120s of the flexible elongate member 120 shifts from a smaller-diameter extended configuration (in FIG. 4A), in which an anchor 110 may move (e.g., slide) with respect to the flexible elongate member 120, to a larger-diameter relaxed configuration (in FIG. 4B) in which the anchor 110 which had been moved is now fixed in place with respect to the flexible elongate member 120. In some aspects, pulling on or longitudinal tensioning of the flexible elongate member 120 causes the shiftable portion 120s to become thinner (typically elongating as well) to have an outer diameter smaller than the inner dimensions of the passage 113 defined in the anchor 110 which is to be moved along the flexible elongate member 120 (which extends through the passage 113). An anchor 110 may thus move along, such as slide along, the length of the shiftable portion 120s when the shiftable portion 120s is in the smaller-diameter extended configuration. Once the force (e.g., pulling, tensioning, etc.) on the flexible elongate member 120 is released, the shiftable portion 120s may revert to a relaxed configuration with a larger-diameter relaxed configuration in which the outer diameter of the shiftable portion 120s is larger than the inner dimensions of the passage 113 defined in the anchor 110. Movement of the anchors 110 along the shiftable portion 120s of the flexible elongate member 120 when the flexible elongate member 120 is in the larger-diameter relaxed configuration is impeded by the outer dimension of the shiftable portion 120s relative to the inner dimensions of passage 113 in the anchor 110. The larger-diameter relaxed configuration of the shiftable portion 120s thus impedes, inhibits, prevents, movement of the anchor 110 relative to (e.g., along the length of) the shiftable portion 120s to maintain the selected position of the anchor 110 along the length of the flexible elongate member 120. Such interaction provides a simpler solution to cinching together tissue engagement members which are coupled together with a flexible elongate member, allowing the members themselves to interact with each other, without the need of an additional step and/or an additional member or element such as a cinch.

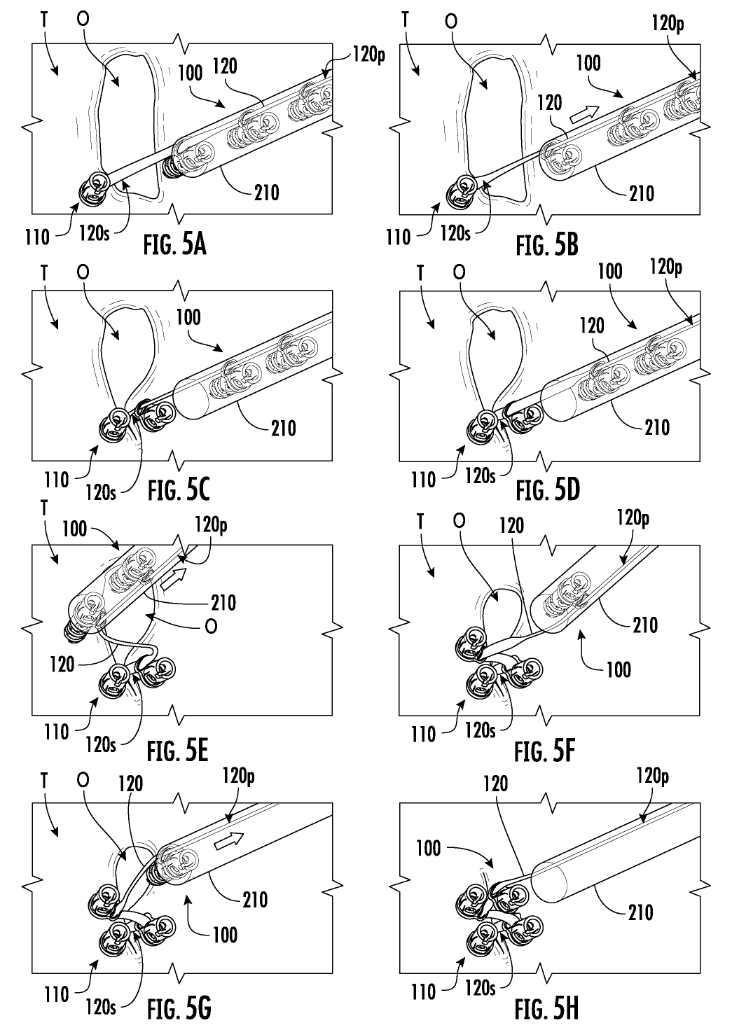

In use, as schematically illustrated in FIGS. 5A-5H, two or more anchors 110 may be engaged with an anatomical treatment site spaced apart from one another, yet coupled together by the flexible elongate member 120. As each anchor 110 after the first-deployed, distalmost anchor is advanced to the anatomical treatment site, the flexible elongate member 120 may be shifted from its larger-diameter relaxed configuration to its smaller-diameter extended configuration to allow the next anchor 110 to be distally advanced towards the next distal anchor 110. The anchor 110 is advanced over the now thinner flexible elongate member 120 and engaged with the desired (e.g., medically indicated) location along anatomical treatment site and with respect to the next distal anchor 110. The flexible elongate member 120 may be pulled after the anchor 110 has been secured in place to cinch together the adjacent anchors 110. The tension / pulling force on the flexible elongate member 120 may then be released to return the shiftable portion 120s of the flexible elongate member 120 to the larger-diameter relaxed configuration to hold the adjacent anchors 110 in place with respect to one another as well as with respect to the flexible elongate member 120 and the tissue T.

In the schematic illustration of FIGS. 5A-5H, the anatomical treatment site is tissue T surrounding an opening O. However, it will be appreciated that a cinching system 100 of the present disclosure may be advantageously used with other configurations of anatomical treatment sites. For instance, although the cinching system 100 is illustrated as closing an opening in tissue, the cinching system 100 may have a variety of other uses, such as reshaping the configuration of an anatomical structure such as a stomach, supporting a weak anatomical structure, fixating an implantable device (e.g., a stent), and other uses generally in conjunction with a medical procedure.

As schematically illustrated in FIG. 5A, a first-deployed / distalmost anchor 110 is engaged with tissue T along an opening O. The delivery device 200 (illustrated in FIGS. 1 and 2) may be used to advance the anchor 110 into the tissue T. For instance, in the illustrated example of an embodiment, the delivery device 200 rotates the anchor 110 to advance distally into tissue. In some aspects, a distal end 222d of an actuator 222 of the delivery device 200 may engage the anchor 110, such as a proximal end 110p of the anchor 110, to actuate and/or advance the anchor 110 to be engaged with (e.g., penetrate or otherwise become anchored or affixed with respect to) anatomical structure such as tissue. For instance, in the case of a helical anchor 110, an actuator 222 of a delivery device 200 may be non-rotatably engaged with the anchor 110 so that rotation of the actuator 222 causes rotation of the anchor 110 to advance into secure engagement with tissue. Schematically illustrated controllers are illustrated in FIGS. 1 and 2 along the control handle 220 for navigating, actuating, etc., various components, features, etc., of the delivery device 200, the exact configuration and operation thereof not being critical to the present disclosure. A non-limiting example of an embodiment of a delivery device usable with a cinching system 100 as described herein is described in U.S. Patent 11,864,751 to Mitelberg, et al., issued Jan. 9, 2024, and titled Endoscopic Tissue Approximation System And Methods, the entire disclosure of which is hereby incorporated herein for all purposes.

As discussed above, the distal end 120d of the flexible elongate member 120 (illustrated in FIGS. 3, 4A, and 4B) is secured with respect to the first-deployed / distalmost anchor 110. For instance, the distalmost end 120dm of the flexible elongate member 120 may be secured with respect to a coupler 112 of the first-deployed / distalmost anchor 110, such as described above. In some aspects, the coupler 112 of any or all of the anchors 110 of the cinching system 100 is rotatable with respect to the associated anchor 110 to facilitate reorientation of the anchor 110 as it is deployed and engaged with anatomical structure such as tissue T. For instance, in the example of an embodiment illustrated in FIGS. 3, 4A, and 4B, the coupler 112 may be formed separately from a proximal end 110p of the anchor 110 and rotatably coupled thereto. More particularly, the coupler 112 may include a ring section 114 from which an eyelet 116 extends (e.g., radially / laterally). The ring section 114 defines an opening 115 rotatably receiving a proximal end 110p of the anchor 110. The eyelet 116 defines the passage 113 through which the flexible elongate member 120 extends. In some aspects, the anchor 110 is formed from a tubular member (e.g., a laser-cut tube), with tabs 116 formed from the wall of the proximal end 110p of the anchor 110 defining a channel 117 in which the ring section 114 of the coupler 112 may be positioned and axially retained along the length of the anchor 110.

Once the first anchor 110 of the cinching system 100 is secured at a treatment site, such as tissue T as illustrated in FIG. 5A, the shiftable portion 120s of the flexible elongate member 120 may then be shifted from the larger-diameter relaxed configuration illustrated in FIG. 5A to the smaller-diameter extended configuration illustrated in FIG. 5B so that the next proximal anchor 110 may be advanced to the schematically-illustrated treatment site. It will be appreciated that a plurality of anchors 110 are schematically illustrated as being advanced through the flexible tubular elongate member 210 of the delivery device 200 at the same time. However, in some aspects, each anchor 110 is separately advanced and deployed before the next anchor 110 is advanced into and distally through the lumen 211 of the flexible elongate member 120. The next anchor 110 is then deployed and engaged with the tissue T, at a deployment site spaced apart from the deployment site of the first anchor 110 (e.g., on the other side of the opening O, across from the deployment site of the first anchor 110), as illustrated in FIG. 5C. The shiftable portion 120s of the flexible elongate member 120 may be cinched (e.g., pulled proximally) to draw together the anchors 110 as well as their respective deployment sites and thereby to close the portion of the opening O across which the first and second anchors 110 have been secured. Pulling or tension on the flexible elongate member 120 may be released to allow the shiftable portion 120s of the flexible elongate member 120 to return to the larger-diameter relaxed configuration, as illustrated in FIG. 5D. The first and second anchors 110 are thereby maintained in the cinched positions / configuration with respect to each other and generally do not move relative to each other or the flexible elongate member 120 because of the increased diameter of the flexible elongate member 120 relative to the passages 113 of the couplers 112 of the anchors 110, such as discussed above.

Additional anchors 110, as medically indicated or necessary, may then be deployed and engaged with tissue T at further deployment sites until the desired effect on the tissue T / opening O is achieved. For instance, the anchors 110 may be sequentially deployed and engaged with tissue T at deployment sites on alternating sides of the opening O in the tissue T. As illustrated in FIG. 5E, a third anchor 110 may be deployed at a deployment site on a side of the opening O opposite the side at which the second anchor 110 had been deployed. A similar process of cinching the shiftable portion 120s of the flexible elongate member 120 to draw together the anchors 110 and their respective deployment sites and thereby to close the portion of the opening O across which the second and third anchors 110 have been secured. As described with respect to the first and second anchors 110, pulling or tension on the flexible elongate member 120 may then be released to allow the shiftable portion 120s of the flexible elongate member 120 to return to the larger-diameter relaxed configuration, as illustrated in FIG. 5F. The second and third anchors 110 are thereby maintained in the cinched positions / configuration with respect to each other and generally do not move relative to each other or the flexible elongate member 120 because of the increased diameter of the flexible elongate member 120 relative to the passages 113 of the couplers 112 of the anchors 110, such as discussed above. Moreover, the larger-diameter relaxed configuration of the shiftable portion 120s of the flexible elongate member 120 also maintains / retains the positions of the second and third anchors 110 relative to the position of the first-deployed anchor 110. In the illustrated example, a fourth and final anchor 110 is delivered and deployed at a deployment site typically spaced apart from the deployment site at which previously-deployed anchors 110 were deployed. The shiftable portion 120s of the flexible elongate member 120 may then be cinched in a manner as described above with respect to FIGS. 5C-5F, reference being made to the above descriptions as applicable mutatis mutandis. In some aspects, only three or four anchors 110 are needed to close an opening O in tissue T. In some aspects, further anchor 110 may be deployed and engaged with the tissue T in a manner such as described above.

Upon deployment of the last anchor 110 in performing the procedure using a cinching system 100 formed in accordance with various principles of the present disclosure, the shiftable portion 120s of the flexible elongate member 120 pulling or other tensioning force on the flexible elongate member 120 is released to leave the shiftable portion 120s of the flexible elongate member 120 in its larger-diameter relaxed configuration, holding the deployed anchors 110 cinched together such as in view of the above descriptions. In some aspects, the coupler 112 (e.g., eyelet 116) of one or more of the anchors 110 may be crimped to maintain / retain the relative positions of the anchor 110 and the shiftable portion 120s of the flexible elongate member 120 for optional added securement. The extent of the flexible elongate member 120 proximal to the cinched anchors 110 may be severed, such as along the transition between the shiftable portion 120s of the flexible elongate member 120 and the proximally-extending portion 120p of the flexible elongate member 120 proximal to the shiftable portion 120s (i.e., along a thinner portion of the flexible elongate member 120). Although the entire length of the flexible elongate member 120 may be shiftable in diameter, in some aspects, only a distal portion of the flexible elongate member 120 is shiftable in diameter. For instance, a length of the flexible elongate member 120 necessary for holding together anchors 110 at the end of the procedure utilizing the cinching system 100 is shiftable in diameter, and proximally extending portions of the flexible elongate member 120 need not be shiftable in diameter. For instance, the shiftable portion 120s of the flexible elongate member 120 may be approximately 2 cm in length from the distalmost end 120dm of the flexible elongate member 120.

It will be appreciated that use of a cinching system 100 formed in accordance with various principles of the present disclosure reduces the need for cinching steps commonly used with current tissue engagement elements used to approximate tissue. Moreover, additional instruments (e.g., cinching instruments) and devices (e.g., cinch anchors or plugs) are not needed as with prior devices or systems, with accompanying risks of the suture inadvertently breaking upon application of cinching forces. Finally, although tensioning of a suture may be performed after each anchor is deployed, such as in prior systems and methods, the anchors of a system as disclosed herein remain approximated better than in prior systems. Tissue approximation with a cinching system of the present disclosure may thus be performed more quickly and with lower risk of delayed procedure (e.g., as a result of a broken suture) than with prior systems and methods.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for approximating anatomical structures, said system comprising:
two or more tissue engagement members each having a distal end configured to be secured to an anatomical structure, and a proximal end; and
a flexible elongate member operably coupled with said two or more tissue engagement members;
wherein:
said two or more tissue engagement members include a distalmost tissue engagement member and one or more tissue engagement members proximal to said distalmost tissue engagement member along said flexible elongate member; and
at least a portion of said flexible elongate member is shiftable between a first configuration in which said one or more tissue engagement members proximal to said distalmost tissue engagement member are operably coupled yet inhibited from moving longitudinally with respect to said flexible elongate member, and a second configuration in which said one or more tissue engagement members proximal to said distalmost tissue engagement member are operably coupled and longitudinally movable with respect to said flexible elongate member.

2. The system of claim 1, wherein said shiftable portion of said flexible elongate member is a distal portion of said flexible elongate member.

3. The system of claim 1, wherein said shiftable portion of said flexible elongate member extends through a passage defined through a portion of one or more of said tissue engagement members.

4. The system of claim 3, wherein:
in the first configuration, said shiftable portion of said flexible elongate member has a larger-diameter relaxed configuration extending through the passage to inhibit relative movement between said one or more of said tissue engagement members and said flexible elongate member; and
in the second configuration, said shiftable portion of said flexible elongate member has a smaller-diameter extended configuration smaller than the passage defined through said one or more of said tissue engagement members to allow longitudinal movement of said one or more tissue engagement members with respect to said flexible elongate member in the second configuration.

5. The system of claim 3, wherein one or more of said tissue engagement members includes a coupler, the passage being defined through said coupler.

6. The system of claim 5, wherein said coupler of at least one of said one or more of said tissue engagement members is rotatably coupled with respect to said at least one of said one or more of said tissue engagement members.

7. The system of claim 1, wherein said shiftable portion is elastomeric.

8. The system of claim 1, wherein said distalmost tissue engagement member is fixedly coupled to a distalmost end of said flexible elongate member so as not to move longitudinally with respect to said flexible elongate member.

9. The system of claim 1, wherein said two or more tissue engagement members are rotatable about said flexible elongate member.

10. The system of claim 1, wherein at least one of said tissue engagement members is a helical tissue anchor.

11. The system of claim 10, wherein said helical tissue anchor includes a coupler rotatably coupled thereto and extending radially from said helical tissue anchor to be operably coupled with said flexible elongate member.

12. A system for approximating anatomical structures, said system comprising:
two or more tissue engagement members each having a distal end configured to be secured to an anatomical structure, and a proximal end;
a flexible elongate member operably coupled with said two or more tissue engagement members; and
a delivery device configured to deliver said two or more tissue engagement members and said flexible elongate member to a treatment site within a patient;
wherein:
said two or more tissue engagement members include a distalmost tissue engagement member and one or more tissue engagement members proximal to said distalmost tissue engagement member; and
at least a portion of said flexible elongate member is shiftable between a first configuration in which said one or more tissue engagement members proximal to said distalmost tissue engagement member are operably coupled yet inhibited from moving longitudinally with respect to said flexible elongate member, and a second configuration in which said one or more tissue engagement members proximal to said distalmost tissue engagement member are operably coupled and longitudinally movable with respect to said flexible elongate member.

13. The system of claim 12, further comprising a medical scope, said delivery device having a flexible tubular elongate member deliverable with a flexible distal end of said medical scope into a patient and advanceable within the patient to a treatment site.

14. The system of claim 12, wherein said one or more tissue engagement members are helical anchors with a sharp distalmost end configured to penetrate tissue, and said delivery device is configured to engage each of said tissue engagement members to rotate each of said tissue engagement members into engagement with tissue and to distally push each of said tissue engagement members out of engagement with said delivery device to deploy said tissue engagement member.

15. The system of claim 14, wherein in the first configuration, said portion of said flexible elongate member is in a relaxed configuration, and after deployment of all of said two or more tissue engagement members by said delivery device, said portion of said flexible elongate member may be cut and left in the first configuration with all of said tissue engagement members inhibited from moving longitudinally with respect to said flexible elongate member.

16. A method of drawing one or more anatomical structures together, said method comprising:
engaging a first tissue engagement member with the anatomical structure at a first deployment site and deploying the first tissue engagement member;
shifting a flexible elongate member, operably coupled with the first tissue engagement member, from a first configuration to a second configuration;
advancing a second tissue engagement member distally along the flexible elongate member and towards the first tissue engagement member;
engaging the second tissue engagement member with the anatomical structure, or another anatomical structure, at a second deployment site spaced apart from the first deployment site and deploying the second tissue engagement member;
cinching the flexible elongate member to draw together the first and second tissue engagement members as well as the deployment sites; and
shifting at least a portion of the flexible elongate member to the first configuration to retain positions of the deployed tissue engagement members with respect to the flexible elongate member.

17. The method of claim 16, wherein the first configuration of the flexible elongate member is a relaxed configuration, said method further comprising releasing force on the flexible elongate member to allow the flexible elongate member to shift to the first configuration to hold the deployed anchors in place with respect to the flexible elongate member.

18. The method of claim 16, further comprising holding the deployed anchors in place with respect to the anatomical structure and to one another by allowing the flexible elongate member to shift to its first configuration, thereby drawing together the one or more anatomical structures.

19. The method of claim 16, further comprising severing an extent of the flexible elongate member proximal to the deployed tissue engagement members, wherein a portion of the flexible elongate member operably coupled with the deployed tissue engagement members is left in the first configuration maintaining drawn together positions of the deployed tissue engagement members with respect to the flexible elongate member.

20. The method of claim 16, further comprising:
advancing one or more additional tissue engagement members distally along the flexible elongate member with the flexible elongate member in the second configuration;
engaging the one or more additional tissue engagement members with respect to the anatomical structure, or another anatomical structure, at one or more further deployment sites spaced apart from the first deployment site and the second deployment site;
deploying the one or more additional tissue engagement members;
cinching the flexible elongate member to draw together the one or more additional tissue engagement members and the first and second tissue engagement members as well as the respective deployment sites; and
shifting at least a portion of the flexible elongate member to the first configuration to retain positions of the deployed tissue engagement members with respect to the flexible elongate member.
